(19) United States
(12) Patent Application Publication
(10) Pub. No.: US 2008/0132914 A1
(43) Pub. Date: Jun. 5, 2008

(54) Device and Method for Removing a Composition From the Skin

(75) Inventors: Isabelle Bossard, Hull (GB); Carl Dibnah, Hull (GB); Andrew Robert McLeish, Sheffield (GB); Tracey Thompson, Hull (GB); Barbara Jenman, Crowle (GB)

Correspondence Address: MERCHANT & GOULD PC, P.O. BOX 2903, MINNEAPOLIS, MN, 55402-0903, US

(73) Assignee: Reckitt Benckiser (UK) Limited, Slough (GB)

(21) Appl. No.: 10/560,191

(22) PCT Filed: Apr. 19, 2004
(86) PCT No.: PCT/GB04/01683

§ 371 Date: May 3, 2007

(30) Foreign Application Priority Data
Jun. 6, 2003 (GB) 0313032.5
Nov. 15, 2003 (GB) 0326651.7

(51) Int. Cl. A61B 17/50

(52) U.S. Cl. 606/131

(57) ABSTRACT

A device for removing a depilatory or other composition from the skin comprises: a non-shaving head (2) which in use is moved over the skin to effect removal of the composition; a handle (4); and a joint (6) between the head and the handle, permitting the head to articulate about the handle.

This invention relates to a device for removing a composition from the skin, and to an associated method. The invention relates in particular, but not exclusively, to a device for use in a depilatory method.

Hair removing compositions are widely available. They may be epilatory, and effect hair, removal by mechanical action. They may be depilatory, and effect hair removal, by causing hair degradation by chemical action.

In the case of depilatory compositions, the user applies these to their skin, leaves them for a predetermined interval to allow them to work, and then removes them from the skin, usually using an article which is provided. This is typically a cloth, sponge or an elastically flexible plastics sheet. Such a plastics sheet may be formed with a curvature so as to facilitate removal of the composition and the hair entrained within it. However some consumers find such a device messy or difficult to use. Many hold such devices close to the leading edge which contacts the skin and consequently it is difficult for them to keep their hand clear of the composition, and hair being removed. Furthermore it can be difficult to reach some areas, such as parts of the backs of legs, using such devices.

We have now devised a different type of device to remove compositions from the skin, including depilatory compositions, efficiently and cleanly.

In accordance with a first aspect of the present invention there is provided a device for removing a composition from the skin, the device comprising: a non-shaving head which in use is moved over the skin to effect removal of the composition; a handle; and a joint between the head and the handle, permitting the head to articulate about the handle.

In this specification when we use terms such as “downward” and “underside” we are referring to the side of the device which faces the skin, in use. Terms such as “upwardly” and “upper” denote the opposite direction. The head is regarded as the front of the device and the tip of the handle as the rear of the device.

A preferred device is designed for use in hair removal; to be drawn over skin to which a depilatory composition has been applied, in order to remove, after any required interval, hair and the depilatory composition. Whilst the device may effect the breaking of some hairs which have been weakened by the depilatory composition it cannot be used as a shaving device. Accordingly it does not have a cutting blade.

Preferably, however, it has the general appearance of a shaving device. Thus it preferably has a generally stick-like or wand-like handle, and a wider head. Preferably the maximum width of the head is 50% greater than the maximum width of the handle, preferably at least 80% greater.

The head suitably has a leading edge which, in use, makes contact with the skin. Preferably this is a straight edge. The leading edge is preferably generally parallel to but maximally displaced from the joint. By parallel to the joint we allude to an axis about which articulation takes place.

Preferably the leading edge is angular but not sharp, to the extent that it might effect cutting, either of skin or hair. Thus, by eye when viewed in magnification it can be seen to be radiused, in preferred embodiments. Preferably the radius of such a leading edge is at least 0.25 mm, more preferably at least 0.5 mm, and most preferably at least 0.65 mm. Preferably the radius of such a leading edge is up to 1.5 mm, more preferably up to 1 mm, and most preferably up to 0.85 mm. It may be a compound radius for example having a major radius in accordance with the definitions given above and a minor radius, smaller than the major radius, at the distal tip of the leading edge. Preferably such a minor radius is up to 0.5 mm, more preferably up to 0.3 mm, most preferably less than 0.25 mm. Preferably it is at least 0.1 mm, more preferably at least 0.15 mm.

The leading edge region of the head may comprise a fin composed of a material which is softer in feel than the material used for the head generally. Preferably, the leading edge comprises a fin of elastomeric material.

Preferably the head is firmly carried by the handle; in the absence of a force or when merely touched there is no tendency for it to be deflected. In use when bearing upon the skin it may articulate against a resistance force.

In use, the user holds the handle and places the leading edge of the head on the skin. Typically the arrangement is such that the head, and the device as a whole, make an acute angle to the skin when the device is in position to be drawn across the skin, in the rearwards direction. The force applied by the head to the skin is provided, by the user to the handle, and this is transmitted by the joint, to the head. Preferably the head may articulate upwardly, so that the usage is still comfortable, and not mechanically aggressive to the skin. The extent of articulation preferably depends on the force applied. Thus the joint is preferably such that when the head is in contact with skin, a downward force applied to the handle is always delivered to the head, whatever position the head has moved to; and in each position of the head it experiences a resistance force from the joint.

Preferably at an extreme position articulation of the head towards the handle is terminated. This may be achieved by provision of a stop means or resilient means within the joint.

Preferably, movement of the head about the handle in the opposite direction, so as to splay open the joint, is limited. This may suitably be by the provision of resilient means within the joint preventing it from articulating excessively.

Suitably resilient means, for example a packing or moulding of an elastomeric material, may be present in the joint such that there is a resilient hindrance to articulation of the head excessively in either direction, about the handle.

Preferably the head articulates about its rear edge region, where it connects to the joint, preferably about its rear edge itself.

Suitably the head is able to articulate relative to the handle (by which we mean from its unflexed or rest position to its maximally flexed—that is, maximally articulated—position when in use) through an angle of at least 1°, preferably, at least 5°, and more preferably at least 10°. Yet more preferably the head is able to articulate relative to the handle through an angle of at least 25°, and most preferably at least 40°.

Suitably the head is able to articulate relative to the handle through an angle of up to 120°, preferably up to 90°, and more preferably up to 75°. Most preferably the head is able to articulate relative to the handle through an angle of up to 60°.

Suitably when the head articulates about the handle the leading edge is able to move through an arc of at least 2 mm, preferably at least 3 mm, more preferably at least 6 mm. Yet more preferably when the head articulates about the handle the leading edge is able to move through an arc of at least at least 10 mm, and most preferably at least 15 mm.

Preferably when the head articulates about the handle the leading edge is able to move through an arc of up to 50 mm, preferably up to 40 mm, and most preferably up to 30 mm.

Preferably the device has a memory property, such that once the force on the head is reduced or removed it is able to recover its previous or rest position. Thus the device may be of a material which is elastic or elastomeric.

We do not exclude embodiments in which the head is separate from the handle. Preferably, however, the device is unitary. A unitary device may be formed of one material or it may be formed of more that one material; set together in the manufacture but thereafter inseparable unless the device is destroyed or damaged. For the handle may be formed of a generally rigid plastics material which carries on at least a part of its outer surface an elastomeric material, for improved feel. The elastomeric material may suitably be formed with grip-assisting formations, for example one or more of fins, ribs, grooves and bumps.

Typically the leading edge is a 20-60 mm line, preferably 30-50 mm.

Preferably the head is curved when viewed from the side. Preferably it is downwardly concave.

Preferably the handle is curved when viewed from the side. Preferably it is downwardly concave.

Preferably the device as a whole is curved, preferably being downwardly concave, with the curvature of the head preferably leading smoothly into the curvature of the handle.

Preferably the device is formed from a plastics material, especially a thermoplastics material. Polyolefins are especially suitable, notably polyethylene (HDPE or LDPE) and, especially polypropylene. Such materials allow the preferred resistive articulation of the head about the handle, allow for elastic recovery, are readily moulded, and are tough, having little tendency to brittle failure, as might otherwise take place within the joint.

Copolymers or polymer blends may be used.

Preferably the device is formed from a plastics material having a Flexural Modulus of at least 0.8 GPa, more preferably in excess of 1 GPa.

Preferably the device is formed of a plastics material having a Flexural Modulus of up to 1.5 GPa, more preferably up to 1.2 GPa.

Preferably the device is formed of a plastics material having a Melt Flow Index of at least 10 g/10 mins, more preferably at least 18 g/10 mins.

Preferably the device is formed of a plastics material having a Melt Flow Index of up to 30 g/10 mins, more preferably up to 25 g/10 mins.

Flexural Modulus measures a material's ability to resist deformation under an applied load. For the purposes of this specification the reference test method is that described in ISO 178.

Melt Flow index (MFI) measures how quickly a thermoplastic material will flow through a known aperture, at a known temperature and under a known pressure. The higher the MFI the faster the material will flow. For the purposes of this specification the reference test method is that described in ISO 1133.

The plastics material may contain one or more additives to improve its properties for the task in hand. For example a plasticizer may be added in order to improve its suppleness or flexibility.

Preferably the device is a unitary plastics moulding. Suitably the joint is achieved by provision of a web of material between the handle and the head, preferably thinner than both.

Preferably the handle is a substantially rigid part. Preferably the head is a substantially rigid part. Preferably, therefore, the articulation of the head about the handle arises substantially wholly because of the nature of the joint; there is substantially no propensity for deflection elsewhere.

Preferably the device weighs less than 12 g, and more, preferably weights less than 8 g.

Preferably the handle is of a waisted shape, having a widened distal region and a widened proximal region (adjacent the joint), with the waist in between. The handle may have a widened distal region which has a non-shaving edge, adapted to effect removal of the composition. The widened distal region is preferably narrower than the head of the device, so that the user can choose from a wider and a narrower edge, for different parts of the body. Preferably the distal region has a depression to aid holding, most preferably on its upper side. Preferably the proximal end region of the handle has a depression on its upper side. In each of these cases the depression could be replaced by a textured non-slip surface, which could be moulded in.

The handle could be a solid piece but more preferably it is generally U-shaped, downwardly open.

The handle could be adapted to release a fragrance. The fragrance could be moulded into any moulded plastics article or could be adhered to the surface of the handle, for example sprayed onto it. The fragrance could, for example, be in the form of fragrance microcapsules which are ruptured, to release the fragrance, when the device is grasped.

Preferably the joint is formed by a web of the plastics material, suitably at the base of a notch which is open in the upwards direction. The notch could be of U-section or V-section. The walls of the notch may be formed by the end faces of the handle and the head. Limitation of flexure in the sense of the head towards the handle may arise simply by the closure of the notch, as by abutment of the facing ends, of the head and the handle. Alternatively the notch may be packed or moulded with an elastomeric material to put limits on the articulation of the head in both directions.

Alternatively, the head may be connected to the handle by a flexible connection of a serpentine form, in which the “windings” are closely pitched. The windings thereof may be packed or moulded with an elastomeric material to put limits on the articulation of the head in both directions.

Preferably the head is bounded by the joint, by the leading edge, which is preferably wider than the joint, and by lateral edges between them. Preferably the lateral edges of the head are convex. The convex edges may have corresponding curved walls upstanding from them, preferably being tallest near to the joint and tapering towards the frontal corners of the head (that is, the junctions between the convex lateral edges and the leading edge). Preferably the head has the appearance of a circle or ellipse truncated by the leading edge.

The junctions between the leading edge of the head and the adjoining edges may be radiused, to reduce the risk of them jagging against the skin.

The device may, in principle, be used to remove any composition from the skin, for example a cleansing or moisturising body pack. However it will be evident from the foregoing that the primary interest is in relation to the removal of a depilatory composition, along with entrained hair.

In accordance with a second aspect of the present invention there is provided a method of removing a composition from the skin, in particular a depilatory composition containing hair, by use of a device of the first aspect.

In accordance with a third aspect of the present invention there is provided a method of effecting depilation, comprising the steps of:

applying a depilatory composition to the skin;

allowing it to remain on the skin for a pre-determined interval;

removing the depilatory composition and depilated hair by moving a device as defined in the first aspect over the skin; and

rinsing the skin.

Preferably the method is one in which, in addition to the depilatory action of the depilatory composition, there is an additional hair removing action of an epilatory nature, achieved by the device. The device is preferably not epilatory in its action in the absence of a depilatory composition; but preferably it is epilatory in its action in conjunction with a depilatory composition, to the extent that hairs weakened by the depilatory composition may be removed by the device. This is potentially very useful because it means that the depilatory composition need only be left on the skin for a reduced period—for example 3 minutes. A longer period would usually be required if the depilatory composition was required to remove all of the treated hair growth, but this would carry with it an increased risk of causing skin irritation. In accordance with the present invention a shorter period can be used in the knowledge that the device can complete the task.

The depilatory composition described herein may for example be a cream, lotion, gel or foam.

The device could be sold on its own. Preferably, however, it is sold in a pack with a receptacle of the composition which is to be applied to the skin. The receptacle may for example be a jar or tube; a conventional aerosol canister; or a multi- or bi-compartment aerosol product (in which the composition and a compressed gas are segregated, inside the canister, the compressed gas driving out the composition when a valve is operated). In the case of a depilatory composition the latter is a preferred way of supplying the composition.

A pack of a composition to be applied to the skin and a device of the first aspect could be a carton with the two components in, or it could be a clear plastics package encompassing them (blister pack, or shrink sleeve). Alternatively, the device of the first aspect could be engaged directly with the receptacle which contains the composition. For example a canister could have a modified cap to which the device is secured.

In accordance with a fourth aspect there is provided a pack comprising a device as defined above and a depilatory composition.

The invention will now be further described, by way of example, with reference to the accompanying drawings, in which:

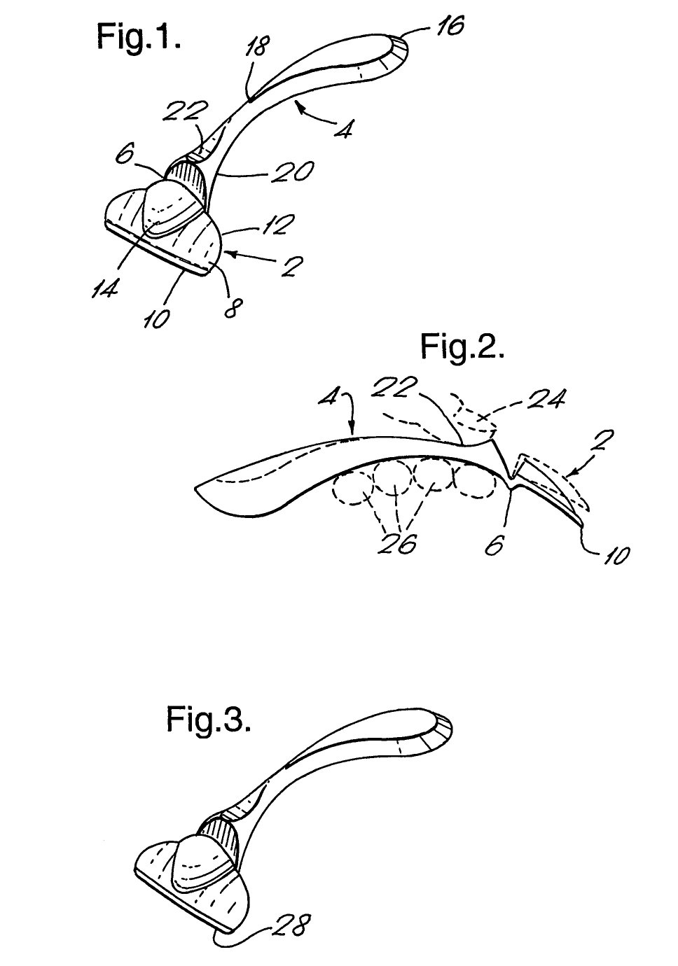

FIG. 1 shows a first embodiment of device, in perspective view;

FIG. 2 shows the device of FIG. 1, in side view;

FIG. 3 shows a second embodiment of a device, in perspective view;

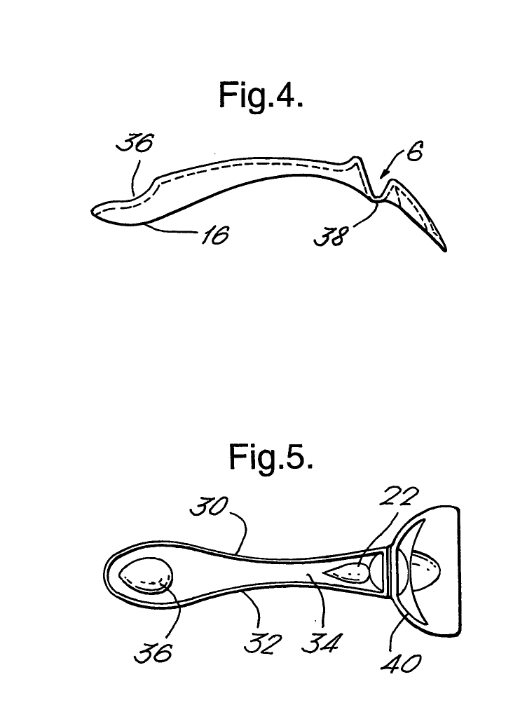

FIG. 4 shows a third embodiment of device, in side view;

FIG. 5 is a view from the underside, of the device of FIG. 4;

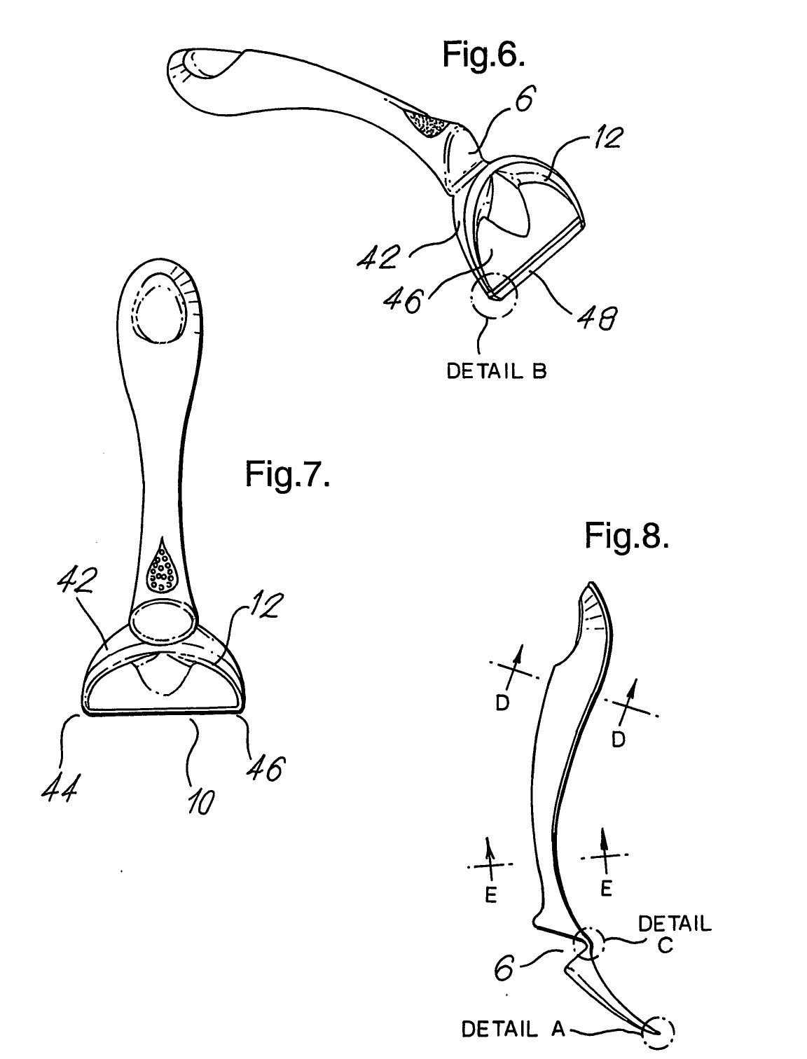

FIG. 6 shows a fourth embodiment of the device, in perspective view;

FIG. 7 shows the device of FIG. 6 in plan view;

FIG. 8 shows the device of FIG. 6 in side view;

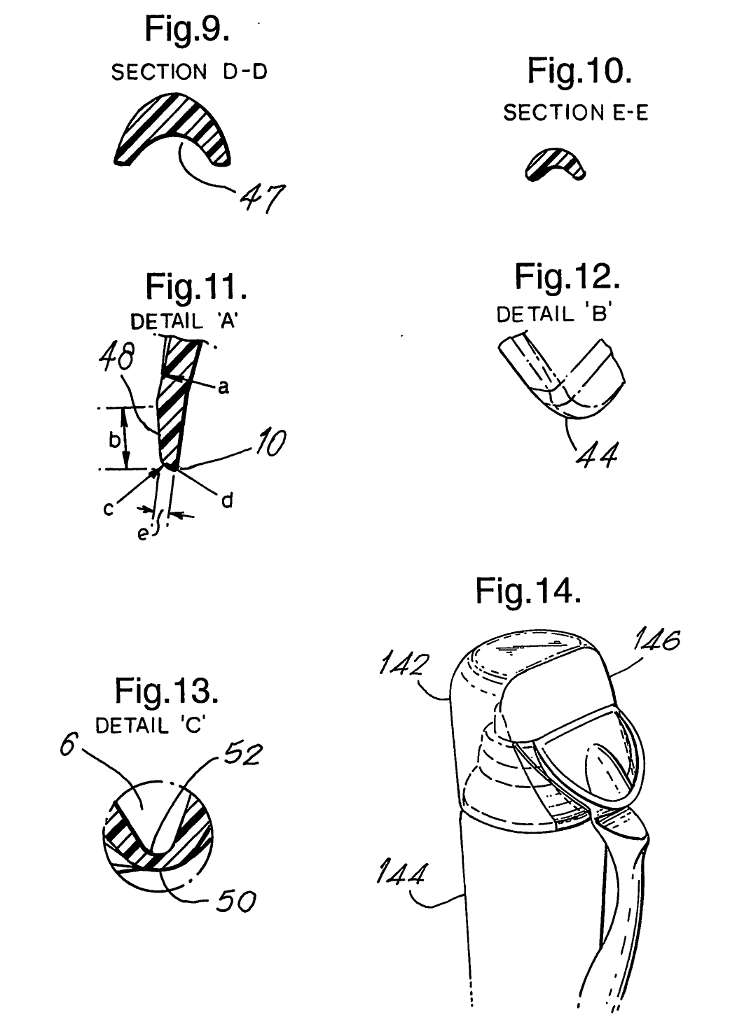

FIG. 9 is a cross-section through line D-D of FIG. 8;

FIG. 10 is a cross-section through line E-E of FIG. 8;

FIG. 11 is an expanded view of Detail A shown in FIG. 8;

FIG. 12 is an expanded view of Detail B shown in FIG. 6;

FIG. 13 is an expanded view of Detail C shown in FIG. 8; and

FIG. 14 shows a device coupled to a canister of a depilatory composition.

Each of the embodiments to be described is intended for use with a depilatory composition.

The device of the first embodiment shown in FIGS. 1 and 2 comprises a wide head 2 and a stick-like handle 4, connected together by a joint 6 which acts as a hinge, permitting the head to articulate about the handle on application of a force, and against a resistance force. It will be seen in FIG. 2 that the head and handle terminate in end faces which join together at their lower edges at the joint 6. The end faces in effect define the joint as a V-shaped notch, upwardly open.

The general appearance of the device is similar to that of a razor device, in having the head 2, which is of similar width to the head of a razor device, and the much narrower stick-like handle 4. However the device is not, in fact, a razor device. It does not have any metallic cutting blade(s).

As can be seen in FIG. 2 the device is generally curved in side view, being concave when considered from the downward direction.

The device is a one-part polyolefin moulding. The head 2 and the handle 4 are both generally rigid, whilst the joint 6 is thinner, and is flexible. Thus the head 2 may articulate or flex relative to the handle 4, the articulation being wholly provided by virtue of the flexibility of the joint 6.

The head 2 has a leading edge region 8, on its opposite side from the joint 6. The leading edge region 8 terminates in a leading edge 10, 44 mm long in this embodiment. The leading edge 10 is formed with a radius of 0.75 mm. This radius means that although good removal of a material from the skin by a scraping action can be achieved, and substantially weakened hairs might be broken and removed, undegraded hairs could not be cut or broken, nor could skin be. Between the leading edge 10 and the joint 6 the head has outwardly curved or bowed side edges 12, such that the overall plan view shape of the head is approaching semi-circular. On its upper face the head has a bulbous part 14. This is present for aesthetic reasons and to impart rigidity to the head.

The handle is of solid plastics material, and is somewhat waisted in shape, having a wide and thick, rounded, distal region 16 of width 22 mm, a narrowed waist 18 of width 10 mm, and a proximal region 20 which is wider than the waist, widening to a maximum width of 18 mm, this being immediately adjacent the joint 6. The upper face of the proximal region 20 is moulded with a non-slip textured portion 22. This can be seen in FIG. 1. As can be seen in FIG. 2 this textured portion rises towards the joint 6. The net result is that the textured portion 22 provides a comfortable location for the thumb 24 of a user, and facilitates application of a force during use. Meanwhile the user's fingers 26 are located on the concave underside of the handle.

In use a depilatory composition (for example a cream, lotion, foam or gel) is applied to the skin, usually by hand, and is left for the required period of time. This is usually five minutes although we favour the use of a depilatory composition which is formulated for removal in three minutes. The device described above is then used to remove the composition, together with depilated hairs. The device is moved rearwardly over the skin, by a drawing action, in the manner of a wet razor. The downward force applied by the user to the handle is transmitted, to the head, whose leading edge is in contact with the skin. The head flexes slightly, to the position shown in dotted lines in FIG. 2, as a reaction to the force acting between the head and the leading edge of the skin. If the user applies a higher downward force the head flexes more. In essence the head is self-correcting, against the risk of pressing too hard against the skin. The articulation of the head relative to the handle takes place against the resistance within the joint. If there were no resistance the head would be floppy on the handle and the device would be of no use.

If an extremely high downward force were applied to the head it could conceivably articulate to its limit position, in which the V-shaped notch has closed; the end face of the handle in effect acting as a stop means. However this is not intended in this embodiment and is not characteristic of any expected use.

The handle could be held in different positions. For example it is designed to be comfortable for a user also to hold the distal end of the handle between thumb and fingers so that the leading edge is well beyond the tips of the fingers. This may be useful when removing material from areas which are difficult to reach, for example the backs of legs.

In the first embodiment the polyolefin is polypropylene but in other embodiments of similar design polyethylene, for example LDPE or HDPE, may be preferred.

The embodiment shown in FIG. 3 differs from that of FIG. 2 only in the provision of an elastomeric fin 28, at the leading edge. It may be of any elastomeric material and may be set into the product in any typical way, for example by dual shot moulding. Preferably it is fairly firm and/or narrow, so that the provision of the joint still provides benefit.

FIGS. 4 and 5 show a third embodiment which is also closely related to the first embodiment. Only the significant differences will be described. In other respects the description of the first embodiment applies to the third embodiment.

A first difference is that the handle is not of solid plastics, but is a downwardly concave moulding. That is, in transverse cross-section it is U-shaped. FIG. 5 is a view from underneath the device and this shows walls 30, 32 and a recess 34 between them. This means that the volume of plastics used in the device can be reduced from about 11 cm³ to about 7 cm³, and provides improved grip.

A second difference is that the distal end 16 of the device is formed with a recess 36. This is to aid the holding of the device at the distal end, as described in relation to FIG. 1. The outline of this recess can be seen in the underside view of FIG. 5, along with the underside of the textured portion 22.

A third difference is in the joint 6, which is not in the form of a V-shaped notch. The end faces of the handle and the head do not meet, as they did in the first and second embodiments. Instead they are joined together by a short bridge 38. The joint is in effect in the form of a U-shaped notch. This gives good flexibility and, because the location of articulation is more diffuse, a lower risk of stress damage.

The underside of the head, adjacent to the joint 6, is formed with a crescent-shaped part 40 which increases its rigidity.

A fifth difference is that the joint is filled with a resilient packing 41, bonded into the joint. The resilient packing limits the flexure of the head about the handle, in both directions, that is, clockwise and anticlockwise.

FIGS. 7 to 13 show a fourth embodiment which is also closely related to the first embodiment. Only significant further details will be described. In other respects the description of the first embodiment applies to the fourth embodiment.

The first difference is that the head has, extending upwardly from its outwardly curved side edges 12, an upstanding wall 42. Although it is not shown clearly in the drawings—but can be seen to some extent in FIG. 6—the wall rises obliquely from the main sheet region 46 of the head, at an angle of approximately 60° thereto. This wall is tallest adjacent to the joint 6, and diminishes or tapers towards the front corners 44, 46, at respective ends of the leading edge 10. At those corners 44,46 the wall 42 disappears.

Like the third embodiment, the fourth embodiment has a handle which is downwardly concave, as shown by FIGS. 9 and 10, which are cross-sections taken through the handle. At section D-D shown in FIG. 9 the maximum width of the handle is 21 mm, and the maximum width of the concavity 47 formed in the underside of the handle is 15 mm. The maximum height of the handle is 13.5 mm and the depth of the cavity is 8 mm. At section E-E shown in FIG. 10 the maximum width of the handle is 11 mm, and the maximum width of the concavity 47 formed in the underside of the handle is 6 mm. The maximum height of the handle is 5 mm and the depth of the cavity is 2 mm.

The device of the fourth embodiment also differs from that of the first embodiment in that the corners 44, 46 at the front of the head are more markedly radiused, to reduce the risk of snagging against the skin. FIG. 12 shows the corner 44 in a detailed view. The radius of each corner is 3 mm in this embodiment.

FIG. 11 shows the leading edge 10 in a detailed cross-sectional view. This is of complex shape. The main sheet region 46 is of thickness 1.5 mm (dimension a). Approaching the edge 10, the upper surface is bevelled as at 48, and the length of this bevel is 3.2 mm (dimension b). The bevel leads into a first radiused region (dimension c) , r=0.8 mm, and this leads in turn to a more sharply radiused region, r=0.2 mm which connects to the under surface of the sheet region 46 (dimension d). The thickness of the head measured at the junction of the bevel 48 and the first radiused region, is dimension e, 0.8 mm.

FIG. 13 is a detailed sectional view of the joint 6. This is made up of a under surface 50 having a radius of 4 mm and an upper surface 52, representing the upwardly open base of the joint, having a radius of 1.5 mm. The thickness of the material at the base of the joint is 1 mm.

The width of the leading edge is 44 mm. The length of the handle, measured up to the mid-point of the joint, is 99 mm. The handle is waisted. At its distal end the maximum width is 23 mm. At its proximal end, immediately adjacent the hinge, its width is 18 mm. In between its “waist” has a minimum width of 10 mm. The overall length of the device is 123 mm.

In this fourth, embodiment the polymer is a polypropylene random copolymer, having the following properties:

### Flexural Modulus 1050 MPa (ISO 178)

### Melt Flow Index: 21 g/10 mins (ISO 1133)

The device shown in FIG. 6 was tested in conjunction with a glycolate-containing depilatory lotion. The lotion was applied to a 50 mm×50 mm test area already marked in pen on the legs of female volunteers, who had allowed their leg hair to grow. It was left for 3 minutes then removed. In Test 1 the lotion was removed by rinsing, without use of the device or any other manual aid. In Tests 2 and 3 the lotion was removed by means of the device, drawn over the skin against the direction (“grain”) of hair growth. The results are as follows:

Test 1:
Before—95 hairs counted in test area
After—88 hairs counted in test area

Test 2:
Before—90 hairs counted in test area
After—no hairs counted in test area

Test 3:
Before—95 hairs counted in test area
After—no hairs counted in test area

FIG. 14 shows a device generally in accordance with the four embodiments described above secured to a canister of a depilatory composition. The canister is not a conventional aerosol canister but is a bi-compartment aerosol canister. Between the removable cap 142 and canister body 144 there is entrapped a flexible plastics part 144 which is formed so as to retain a device in accordance with the invention. The whole assembly is enveloped in a plastics film, for example as a blister pack. An advantage of this arrangement is that there is no possibility of rotation of the assembly prior to purchase. In an alternative arrangement in which these two parts are within one package, but in separate compartments, there would be a risk that the canister would turn within the package, due to its cylindrical shape. If this happened the presentation to the customer at the point of sale would be spoilt.

In another embodiment a similar result is achieved by using two moulded transparent plastics shells which are engaged together to envelop a canister and a device as described above, the shells together defining a cavity for snug receipt of the canister, and a cavity for the snug receipt of the device. The cavities can be separate but are preferably interconnected, such that the device is nested against the canister, substantially as shown in the FIG. 14 embodiment.

1. A device for removing a composition and depilated hair u from the skin, the device comprising: a non-shaving head which in use is moved over the skin to effect removal of the composition; a handle having a curvature profile and a broadened end distal from the non-shaving head; and a joint between the head and the handle, permitting an articulation of the head about the handle.

2. The device as claimed in claim 1, wherein there is a resistance force acting against articulation of the head, which resistance force increases as the articulation increases.

3. The device as claimed in claim 1, wherein the head comprises an articulation about the handle through an angle in the range of 10 to 40°.

4. The device as claimed in claim 3, wherein when the force, causing articulation of the head, is reduced or removed the head is able to recover a previous or original position.

5. The device as claimed in claim 3, the device being unitary.

6. The device as claimed in claim 5, the device being a one-part device comprising a plastic.

7. The device as claimed in claim 3, wherein the handle is substantially rigid, the head is substantially rigid, and the joint is the only source of the articulation.

8. The device as claimed in claim 3 wherein the joint is formed by a web of plastic material at the base of an upwardly open notch between the handle and the head.

9. (canceled)

10. (canceled)

11. (canceled)

12. (canceled)

13. (canceled)

14. A device for removing a depilatory composition and depilated entrained hair from skin, the device comprising a translucent, integrally molded thermoplastic unit, the unit consisting essentially of:
a. a non-shaving head, the head comprising a planar surface comprising a flat top surface and a flat under surface, the planar surface ending in a single straight edge;
b. a handle having a curvature profile and a broadened end distal from the head; and
c. a single joint between the handle and the head providing limited articulation of the head with respect to the handle.

15. The device as claimed in claim 14, wherein in the limited articulation of the head there is a resistance force acting against articulation of the head, which resistance force increases as the articulation increases.

16. The device of claim 14, wherein the joint is formed by a web of plastic material at the base of an upwardly open notch between the handle and a curved surface of the head.

17. (canceled)

18. (canceled)

19. (canceled)
